(19) United States
(12) Patent Application Publication
(10) Pub. No.: US 2024/0327773 A1
(43) Pub. Date: Oct. 3, 2024

(54) ORGANOID MICROINJECTION SYSTEM AND APPARATUS

(71) Applicant: Merck Sharp & Dohme LLC, Rahway, NJ (US)

(72) Inventors: Jason A. Cassaday, Coppersburg, PA (US); Erik C. Hett, Arlington, MA (US); Stephen H. Kasper, Watertown, MA (US); Linda Adelle Lieberman, Weston, MA (US); Sina Mohammadi, Watertown, MA (US); Carolina D. Morell-Perez, Fairfax, VA (US); Clifford Sampson, Gaithersburg, MD (US); Brian Andrews Squadroni, Philadelphia, PA (US)

(73) Assignee: Merck Sharp & Dohme LLC, Rahway, NJ (US)

(21) Appl. No.: 18/687,322

(22) PCT Filed: Sep. 7, 2022
(86) PCT No.: PCT/US2022/042672

§ 371 Date: Feb. 28, 2024

Related U.S. Application Data
(60) Provisional application No. 63/243,838, filed on Sep. 14, 2021.

(51) Int. Cl. C12M 1/26; C12N 5/00

(52) U.S. Cl. CPC C12M 33/04; C12N 5/0062

(57) ABSTRACT

A microinjection system and apparatus comprising stationary trough chips having wells and troughs to allow for efficient organoid microinjections are described.

### BACKGROUND OF THE INVENTION

### (1) Field of the Invention

The present disclosure relates generally to an organoid microinjection system and apparatus. More specifically, the present disclosure relates to stationary trough chips having wells and troughs to allow for efficient organoid microinjections, particularly when integrated with an imaging and tracking system.

### (2) Description of Related Art

The development of 3D gut organoids has allowed for the study of physiologically informative outcomes of the gut epithelium in response to stimuli. The fully enclosed hypoxic lumen of these 3D structures allows the unique opportunity to microinject into the lumen, access the apical surface of the gut and deliver the stimulus of interest (e.g. bacteria, antibody, small molecule, damage/repair agent, etc.) and/or a therapeutic modality. Due to the size of the organoids, microinjections should be precise and accurate. This process can be laborious and inefficient. Additionally, the injected organoids must be properly tracked to obtain helpful data.

Williamson et al. reported a high-throughput organoid microinjection platform to study gastrointestinal microbiota and luminal physiology, Cell Mol Gastroenterol Hepatol. 6:301-319 (2018). Of interest are Kassis et al., OrgaQuant: Human Intestinal Organoid Localization and Quantification Using Deep Convolutional Neural Networks. Sci Rep. 9:12479 (2019); Hill et al., Bacterial Colonization Stimulates A Complex Physiological Response In The Immature Human Intestinal Epithelium, Elife 6: e29132 (2017); Bartfeld et al., In Vitro Expansion Of Human Gastric Epithelial Stem Cells And Their Responses To Bacterial Infection, Gastroenterology. 148:126-136 (2015); Engevik et al., Human Clostridium Difficile Infection: Altered Mucus Production And Composition, Am J Physiol Gastrointest Liver Physiol. 308: G510-G524 (2015); and Heo et al., Modelling Cryptosporidium Infection In Human Small Intestinal And Lung Organoids, Nat Microbiol. 3:814-823 (2018).

There exists a need for devices that improve upon and advance the methods of organoid microinjections.

### BRIEF SUMMARY OF THE INVENTION

The present invention provides a microinjection system for facilitating microinjection of substances into organoids. The system comprises a stationary trough chip to hold organoids in a set configuration. The system further comprises an apparatus comprising an X-Y stage moveable in X-Y planes for receiving the stationary trough chip, a video camera movable in a Z plane positioned above the stage for recording a video stream of the stationary trough chip on the X-Y stage, a microinjector comprising a capillary needle moveable in X-Y-Z planes positioned above the stationary trough chip for injecting substances into the organoid, and software to automate the microinjection process. The stationary trough chip provides an improvement to the methods of organoid injections and overcomes technical challenges present using prior art methods. Substances for injecting into organoids include but are not limited to drugs, chemical molecules, proteins, peptides, nucleic acids, microbes, microbial material, therapeutic modalities, and the like.

Thus, the present invention provides a stationary trough chip for microinjection of organoids which comprises a body including a series of wells, each of the wells having a plurality of troughs disposed therein.

In particular aspects, the body includes a series of wells arranged in a plurality of rows and a plurality of columns. In a further embodiment, the plurality of rows includes two rows of wells and the plurality of columns includes three columns of wells, which further comprises rows of troughs. In particular aspects, the rows of troughs comprise three troughs of equal length. The plurality of troughs is configured and arranged to receive multiple organoids.

In further embodiments, each of the plurality of troughs is approximately 500 μm deep. In a further embodiment, each of the plurality of troughs is approximately 600 μm in length. In a further embodiment of the microinjection system, each of the plurality of troughs is approximately 500 μm deep and approximately 600 μm in length. In a particular embodiment, the trough dimensions may be about 600 μm width×500 μm depth×6 mm length. In further embodiments, at least one of the plurality of troughs contains a semi-solid substrate structure comprising organoids.

In particular aspects, the body comprises polydimethylsiloxane. In further aspects, a glass slide is coupled to the body.

The present invention further provides a method of organoid microinjection, comprising (a) providing a microinjection system comprising an X-Y stage, a capillary needle having a tip containing an injectable substance, and a video camera positioned above the X-Y stage; (b) providing a stationary trough chip comprising a body including a plurality of wells arranged in rows and columns, each of the plurality of wells having a plurality of troughs and a plurality of organoids disposed in at least one of the troughs; (c) placing the stationary trough chip on the X-Y stage; (d) selecting a first organoid in a first trough of the stationary trough chip; (e) moving at least one of the X-Y stage and the capillary needle to align the tip of the capillary needle with the first organoid; and (f) piercing the first organoid and injecting it with the injectable substance.

In a further embodiment of the method, the step of moving at least one of the X-Y stage and the capillary needle to align the tip of the capillary needle with the first organoid includes moving the X-Y stage along a plane in which the stationary trough chip is disposed.

In a further embodiment of the method, the step of moving at least one of the X-Y stage and the capillary needle to align the tip of the capillary needle with the first organoid includes moving the capillary needle along a plane in which the stationary trough chip is disposed.

In a further embodiment of the method, step (e) comprises moving the X-Y stage to bring a first organoid into view of the video camera and moving the capillary needle to align the tip of the capillary needle with the first organoid.

In a further embodiment of the method, a step of selecting a second organoid in the first trough of the stationary trough chip, moving at least one of the X-Y stage and the capillary needle to align the tip of the capillary needle with the second organoid, piercing the second organoid with the tip of capillary needled, and injecting it with the substance. In particular embodiments, the second organoid is injected with the same injectable substance that was injected into the first organoid. In particular embodiments, the second organoid is injected with a second injectable substance, which is different from the injectable substance injected into the first organoid.

In further embodiments of the method, the method comprises a step of logging information on the first substance and the location of the first organoid.

In a further embodiment of the method, providing a stationary trough chip comprising a body includes providing a stationary trough chip having six wells, which in further embodiments comprises a body having wells with each well including three troughs aligned in a column.

The present invention further comprises a microinjection system comprising (a) a stationary trough chip comprising a series of wells, each of the wells having a plurality of troughs disposed therein; (b) an apparatus having an X-Y stage having for receiving the stationary trough chip, the stage being translatable along an X axis and a Y axis defining an X-Y plane; (c) a capillary needle having a tip and a lumen therethrough open at both ends, which is positioned above the stationary trough chip; and, (d) a video camera positioned above the X-Y stage and capable of recording a video stream comprising a frame view of a trough and the tip of the capillary needle, the video camera translatable along a Z-axis normal to the X-Y stage.

In particular embodiments of the microinjection system, the body includes a series of wells arranged in a plurality of rows and a plurality of columns. In a further embodiment, the plurality of rows includes two rows of wells and the plurality of columns includes three columns of wells, which further comprises rows of troughs. In particular aspects, the rows of troughs comprise three troughs of equal length. In particular aspects, the plurality of troughs is configured and arranged to receive multiple organoids.

In further embodiments, each of the plurality of troughs is approximately 500 μm deep. In a further embodiment, each of the plurality of troughs is approximately 600 μm in length. In a further embodiment of the microinjection system, each of the plurality of troughs is approximately 500 μm deep and approximately 600 μm in length. In a particular embodiment, the trough dimensions may be about 600 μm width×500 μm depth×6 mm length. In further embodiments, at least one of the plurality of troughs contains a semi-solid substrate structure comprising organoids.

In further embodiments of the microinjection system, the capillary needle is provided by a micromanipulator comprising a capillary holder having a distal end and a proximal end and a lumen therethrough open at both ends in which the capillary needle is releasably gripped by the distal end of the capillary holder and wherein the micromanipulator enables the translation of the capillary needle along the X, Y, and Z axes.

In further embodiments of the microinjection system, an injection tubing having a distal end and a proximal end is provided wherein the distal end of the injection tubing is releasably gripped by the proximal end of the capillary holder and the proximal end of the injection tubing is releasably connected to a microinjector capable of providing precise and reproducible injections of femtoliter to microliter amounts of fluids.

The present invention further comprises a microinjection system comprising an apparatus having an X-Y stage, the stage being translatable along an X axis and a Y axis defining an X-Y plane; a capillary needle having a tip and a lumen therethrough open at both ends; and, a video camera positioned above the X-Y stage and capable of recording a video stream comprising a frame view of X-Y stage, the video camera translatable along a Z-axis normal to the X-Y stage, the improvement comprising a stationary trough chip comprising a series of wells, each of the wells having a plurality of troughs disposed therein for receiving a multiplicity of organoids, wherein the organoids are positioned within the trough in a row on the same plane and the stationary trough chip is positioned on the X-Y stage.

In further embodiments, each of the plurality of troughs is approximately 500 μm deep. In a further embodiment, each of the plurality of troughs is approximately 600 μm in length. In a further embodiment of the microinjection system, each of the plurality of troughs is approximately 500 μm deep and approximately 600 μm in length. In a particular embodiment, the trough dimensions may be about 600 μm width×500 μm depth×6 mm length. In further embodiments, at least one of the plurality of troughs contains a semi-solid substrate structure comprising organoids. In a further embodiment of the microinjection system, the capillary needle is provided by a micromanipulator comprising a capillary holder having a distal end and a proximal end and a lumen therethrough open at both ends in which the capillary needle is releasably gripped by the distal end of the capillary holder and wherein the micromanipulator enables the translation of the capillary needle along the X, Y, and Z axes.

In a further embodiment of the microinjection system, an injection tubing having a distal end and a proximal end is provided wherein the distal end of the injection tubing is releasably gripped by the proximal end of the capillary holder and the proximal end of the injection tubing is releasably connected to a microinjector capable of providing precise and reproducible injections of femtoliter to microliter amounts of fluids.

### BRIEF DESCRIPTION OF THE DRAWINGS

Various embodiments of the presently disclosed microinjection systems and components are disclosed herein with reference to the drawings, wherein:

FIG. 1 is a schematic perspective view of a microinjection system according to one embodiment of the present disclosure.

FIGS. 2A-B are schematic perspective views of a stationary trough chip according to one embodiment of the present disclosure. In FIG. 2B, W refers to well, T refers to trough, and P refers to position.

FIG. 3 is a schematic perspective view of the stationary trough chip of FIG. 2 disposed on a stage that is ready for microinjection.

FIG. 4 is a schematic view of a capillary holder and capillary needle.

FIGS. 5A-E are screenshots of magnified organoids disposed within a trough of a stationary trough chip for microinjection.

Various embodiments will now be described with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to microinjector systems, conventional methods suffer from some shortcomings as discussed above. Therefore, is a need for further improvements to the devices and methods used for organoid microinjection. Among other advantages, the present disclosure may address these needs.

The development of 3D gut organoids has allowed for the study of physiologically informative outcomes of the gut epithelium in response to any number of stimuli. The fully enclosed hypoxic lumen of these 3D structures allows the unique opportunity to microinject into the lumen of the organoid, accessing the apical surface of the gut and delivering the stimulus of interest (e.g. bacteria, antibody, small molecule, damage/repair agent, etc.). Our initial studies microinjecting these 3D structures revealed technical challenges that we have now overcome by designing a chip-based device to hold the organoids in a set configuration and software to automate the microinjection process. Thus, the present invention provides an improvement in the field of microinjector systems, particularly for the use of injecting organoids.

The present invention provides a stationary trough chip for microinjection, which includes a body including a series of wells arranged in columns and/or rows, each of the wells having a plurality of troughs for receiving at least one organoid. The stationary trough chip may be used in a microinjection system comprising (a) a stationary trough chip comprising a series of wells, each of the wells having a plurality of troughs disposed therein; (b) an apparatus having an X-Y stage having for receiving the stationary trough chip, the stage being translatable along an X axis and a Y axis defining an X-Y plane; (c) a capillary needle having a tip and a lumen therethrough open at both ends, which is positioned above the stationary trough chip; and, (d) a video camera positioned above the X-Y stage and capable of recording a video stream comprising a frame view of a trough and the tip of the capillary needle, the video camera translatable along a Z-axis normal to the X-Y stage.

The microinjection system enables a method of organoid microinjection, comprising (a) providing a microinjection system comprising an X-Y stage, a capillary needle having a tip containing an injectable substance, and a video camera positioned above the X-Y stage; (b) providing a stationary trough chip comprising a body including a plurality of wells arranged in rows and columns, each of the plurality of wells having a plurality of troughs and a plurality of organoids disposed in at least one of the troughs; (c) placing the stationary trough chip on the X-Y stage; (d) selecting a first organoid in a first trough of the stationary trough chip; (e) moving at least one of the X-Y stage and the capillary needle to align the tip of the capillary needle with the first organoid; and (f) piercing the first organoid and injecting it with the injectable substance.

The following provides a further understanding of the present invention.

### Organoid Microinjection Automation System (OMAS)

The system of the present invention provides automated assistance to the scientist for injection of organoids or tissue slices to allow for faster, more reliable injections. The system maintains a detailed log of objects injected as well as injection parameters to leverage as a reference or to perform multiple injections of the same object.

OMAS has an X-Y stage having an LED backlight for holding and illuminating objects for injection. A video camera with is mounted above the X-Y stage on a single axis motor to provide focus on a region of the labware stage approximately 4×3 mm. A micromanipulator (e.g., Eppendorf InjectMan®) comprising a capillary needle is mounted such that the tip of the capillary needle is within the view of a video camera frame. The X-Y stage has two-axis motorized movement, which is utilized to bring different regions of the X-Y stage into camera view and range of the microinjector for injecting an injectable substance. In an exemplary embodiment, the movement of the X-Y stage may be controlled using a computer comprising software written in National Instruments Lab VIEW® programming language.

For organoid injection, a stationary trough chip was designed containing “troughs” in which the organoids are seeded. This layout organizes the organoids in a predictable manner, where they can grow single file while resting on the same Z-plane thus facilitatinginjection. For the stationary trough chip design, a mold or chip was designed with six wells, each well containing three micro-troughs. The troughs may be approximately 500 μm deep. In a further embodiment, each of the plurality of troughs is approximately 600 μm in length. In a further embodiment of the microinjection system, each of the plurality of troughs is approximately 500 μm deep and approximately 600 μm in length. In a particular embodiment, the trough dimensions may be about 600 μm width×500 μm depth×6 mm length. The troughs may be approximately 600 μm wide by 500 μm deep, which are suitable dimensions for containing organoids for growth and injection. Organoids are seeded into the micro-troughs in a semi-solid substrate that provides structure for organoid growth and keeps the organoids in a static position, e.g., a semi-solid substrate such as Corning Matrigel®. This is enables injection tracking as individual organoids can be tracked following injection or through a series of injections over time.

The stationary trough chip is set on the LED backlight mounted to a motorized X-Y axis labware stage. A video camera is mounted above the chip on a Z-axis motor, which provides control for camera focus. A commercial micromanipulator (e.g., Eppendorf Injectman®) is mounted such that the tip of the capillary needle is within the view of the video camera view frame. The X-Y labware stage is utilized to move stationary trough chip such that different wells and troughs come into video camera view while the view frame of the camera and microinjector remain in position above the stationary trough chip.

Software control may be implemented by a computer using a program for controlling the individual modules (e.g., video camera focus, micromanipulator motion and injection, X-Y labware stage motion) and a semi-automated microinjection routine. The OMAS software is an exemplary computer program that enables a user to coordinate the movement and focus of the video camera, movement of the X-Y labware stage, and microinjector movement and injection. For this coordinated routine, the user frames out the target organoid on the video camera feed window of a computer monitor and triggers the injection. A series of movements for the capillary needle are calculated and executed to approach, pierce, inject, and depart the organoid. Information related to the location and size of the organoid, as well as injection parameters, are logged to a database for future reference or recall.

### Software Architecture

The software control for OMAS is built around modules, which control the different components of the system at a low level, with an overarching instrument driver coordinating the execution of the modules. The modules consist of a video stream, motion control, micromanipulator (e.g., Eppendorf Injectman®) control, a system calibration routine, and tracking database. The user can manipulate the different modules and perform “point-and-click injections) using the instrument driver graphical user interface (GUI). The OMAS software was coded with National Instruments Lab VIEW® programming language and libraries.

### Video Stream

Central to the OMAS instrument is the video camera system, which provides a live view of the injection needle over the organoid trough. National Instrument (NI) Vision modules are utilized to collect frames from a gigabit ethernet camera and update the display in real time. This frame collection is handled asynchronously, meaning it continues to update regardless of the state of other modules. Additionally, function is provided to save images at any time either manually by the user or through a programmatic call. One area where this is leveraged is during injection, where an image is automatically captured at the time of injection. These images can be referenced later to confirm the needle pierced the desired target.

### Motion Control

Festo® motors integrated with OMAS handle two main functions of the injection system; labware (e.g., stationary trough chip) positioning with the X-Y labware stage and camera focusing with the Z-axis. Festo® motor controller communications are handled through a controller program such as the NI Modbus library and low-level control (homing, move to position, etc.) was created in conjunction with Biomated Solutions engineering firm.

A flexible framework was then devised for defining and saving motor locations and types of labware for use in the system. For the stationary trough chip, all the necessary dimensions are provided to define the well (and trough in this case) dimensions in order to accurately move different areas of the stationary trough chip into view of the camera frame. In the case of the stationary trough chip, which is molded and fixed to a glass slide, there are small variations in the placement and thickness of the molded portion. To account for this variance, each piece of injection stationary trough chip is registered for use with a base labware type and the slight differences in positioning and focus-height can be offset and stored for that individual unit. With this location and labware infrastructure in place, pivoting to other types of injection paradigms on the platform, such as a tissue slice on a microscope slide, can be done rapidly.

### Microinjection Control

The Eppendorf® Injectman® is an exemplary commercially available micromanipulator, which provides three-axis movement of the capillary needle, and the Eppendorf® FemtoJet 4i® is an exemplary commercially available microinjector, which provides a precision pressure unit for driving the liquid injection of a preselected amount under a preselected force. A joystick is provided to manually drive and inject with the capillary needle. For OMAS, however, we leverage the application programming interface (API) provided by Eppendorf® to automate control through commands sent over serial communications. Full functionality of this instrument is available through the command interface along with feedback providing status and error information. Injection parameters such as pressure and length of injection as well as capillary needle movement speeds and target location can be set dynamically. With this control in place, a complex series of coordinated capillary needle movements and injections are possible which would be difficult or even impossible to do manually.

### System Calibration Routine

The key concept of OMAS is to always have the capillary needle positioned in the frame of the video camera. The X-Y labware stage is first moved to a pre-defined trough location into video camera view, then the micromanipulator motion controller handles movement of the capillary needle to approach and inject any target which is “in frame” with the video camera view. In order to automatically move the capillary needle tip to a desired target in view, a relationship must be established between the micromanipulator motor coordinates and the pixel location of the target on the video stream displayed on a video monitor.

A grid calibration routine was established to provide this relationship. To perform this calibration, the user drives the capillary needle to a set location in the top-left of the image and starts the routine, which is then handled by the software. An image is captured, and the microinjector motor coordinates are saved for this location. A cycle begins where the capillary needle is moved a set number of motor steps in the X and Y plane, where again an image is captured and coordinates stored, until a grid has been traced across the viewable image. The pixel location of the capillary needle tip in each captured image is then determined using an image analysis function. At this point, a grid has been generated with the pixel location and motor coordinates of the capillary needle at points across the image. A target pixel location can then be translated into motor coordinates for the needle through interpolation of the nearest grid points.

### Tracking Database

An SQLite relational database is embedded with the software for tracking stationary trough chips registered on the system as well as information pertinent to each injection performed. As previously mentioned, individual stationary trough chips are registered in the system with a unique identifier and this unit information is stored in the database. When an injection is performed on this unit, an “organoid” object is registered in the database, linked to that stationary trough chip, and containing the necessary information to log its location (well, trough, image pixel location). An “injection” object is also created, linked to the organoid, containing all microinjector settings (capillary needle pressure, injection time, etc.), material injected, a path to the captured injection image, as well as a timestamp.

This tracking information can be leveraged to return to a specific organoid for automated imaging observation or re-injection of organoids of interest. Additional injections within a 50×50 pixel range of a registered organoid are considered to be injections of the same organoid and are registered as such. This tracking capability offers a significant advantage over manual injection.

### “Point-and-Click” Injections

The user can perform an injection routine using the controls and video stream presented in the software. An oval tool is available for circling a region of interest on the video and this is leveraged to outline the target organoid. When an injection is then triggered, the oval center position and mean radius are obtained from the outlined organoid and utilized to generate a series of injection movements. The capillary needle first moves to a safe plane above the stationary trough chip surface and then moves to location from where it can make a straight line move toward the organoid target. The capillary needle then does an approach move to the surface of the organoid target, followed by a final, generally much faster, piercing move to the organoid target center. The movement speeds for each step are configurable in the software to adjust for better piercing efficiency. At this point, the injection is triggered and the movements are reversed to bring the capillary needle back up to the safe plane, ready for further injections.

### Stationary Trough Chip

For the stationary trough design, a mold or trough chip was designed with six wells, each well containing three micro-troughs. The troughs are 600 μm wide and 500 μm deep, ideal dimensions for containing contain organoids for growth and injection. Organoids are seeded into the troughs in a semi-solid substrate that provides structure for organoid growth and keeps the organoids in a static position, e.g., a semi-solid substrate such as Corning Matrigel®. This enables injection tracking as individual organoids can be tracked following injection or through a series of injections over time.

### Exemplary Embodiment

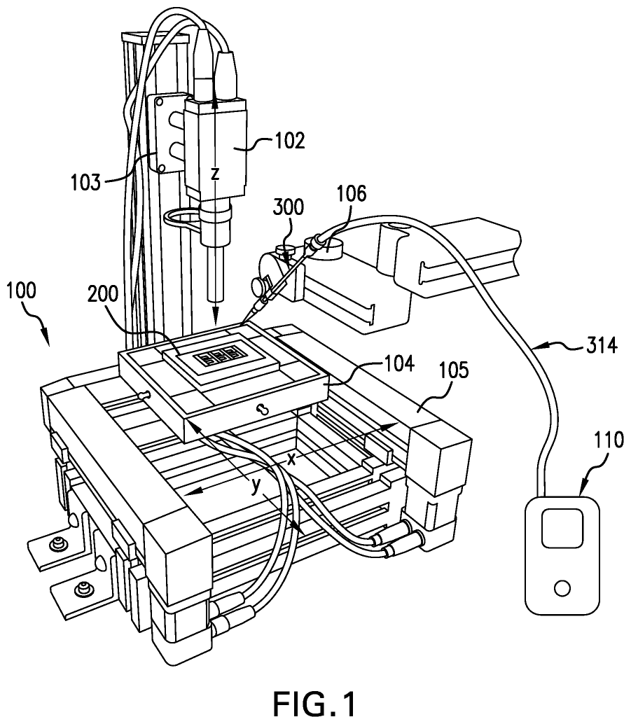

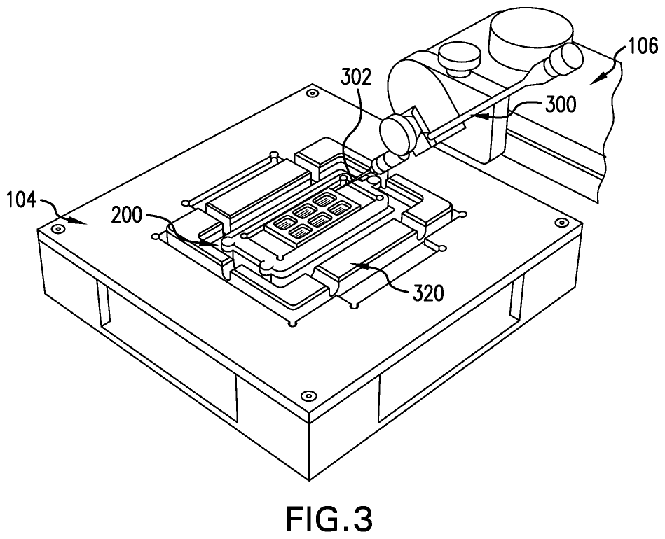

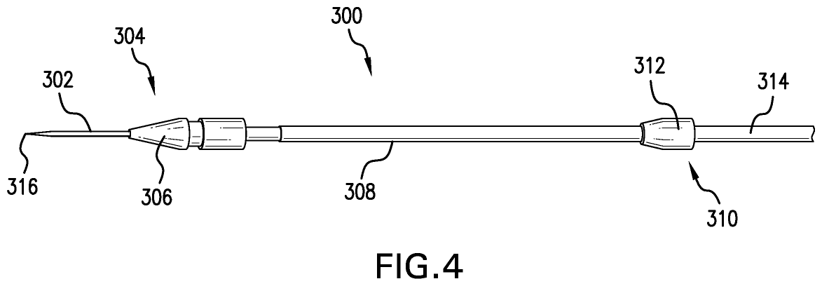

Reference is now made to FIG. 1, which shows an exemplary embodiment of microinjection system 100. Microinjection system 100 generally includes a stationary trough chip 200, video camera 102 (e.g., a camera with 4× lens), the video camera 102 being coupled to a moveable arm 103 that may translate along the z-axis so that the camera 102 can move up (i.e., away from a specimen) and down (i.e., toward the specimen). Movement of the camera 102 along the z-axis may be used to properly focus the camera of the specimen. As shown, camera 102 is disposed above a light source (e.g., LED backlight) and a motorized X-Y stage 104 (e.g., in this example, a Lab VIEW® controlled X-Y stage), the X-Y stage 104 being translatable via a motorized platform 105 in directions orthogonal to the camera along an x-axis (i.e., side-to-side) and/or a y-axis (i.e., front and back). A micromanipulator 106 comprising a capillary needle holder 300 (see also FIG. 3) for holding a capillary needle 302 (as shown in FIG. 3 and FIG. 4) and comprising a lumen therethrough for injecting an injectable substance is shown disposed adjacent to X-Y stage 104 so that the capillary needle 302 (as shown in FIG. 3 and FIG. 4) is close to and above the X-Y stage 104 having disposed thereupon a stationary trough chip 200 (see also FIG. 3). The micromanipulator may be under motorized control so as to translate along the X, Y, and Z axes. In this example, micromanipulator 106 is an Eppendorf® InjectMan®, although it will be understood that other suitable injection systems may be used. In general, the microinjector and micromanipulator are electronically connected, which allows synchronization of the injection movement of the micromanipulator and the injection pressure of the microinjector. This semi-automated step may be triggered by one click onto the joystick key of the manipulator. A commercially available microinjector 110 capable of providing precise and reproducible injections of femtoliter to microliter amounts of fluids to organoids, cells, or cell nuclei (e.g., Eppendorf® FemtoJet 4i®) is connected to the capillary needle holder 300 via injection tubing 314 for controlling injection of an injectable substance.

As shown in FIG. 3, the micromanipulator 106 comprises a capillary needle holder 300. As shown in FIG. 4, the capillary needle holder 300 comprises a tubular member 308 having a distal end 304 and a proximal end 310 and a lumen therethrough, a capillary grip 306 positioned at the distal end 304 and an injection tubing grip 312 positioned at the proximal end 310 for attachment of injection tubing 314 from a microinjector 110 capable of effecting delivery of small quantities (e.g., in the femtoliter to microliter range) of injectable substances from a capillary needle 302 to cells, organoids, and the like, at the appropriate injection pressure. The capillary holder 300 further comprises a capillary needle 302 having a proximal end and a distal end tip 316 with a lumen opened at both ends extending therethrough wherein the proximal end of the capillary needle 302 is inserted into the capillary grip 306 which reversibly grips the proximal end of the capillary needle 302. When the capillary holder is assembled with the capillary needle 302 and tubing 314, a continuous opening is provided from the microinjector through the injection tubing and capillary holder to and through the capillary needle 302.

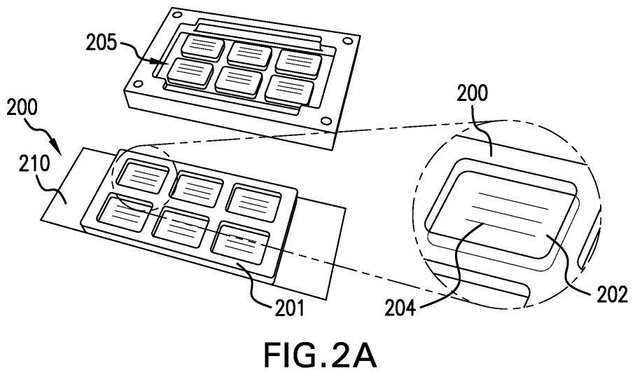

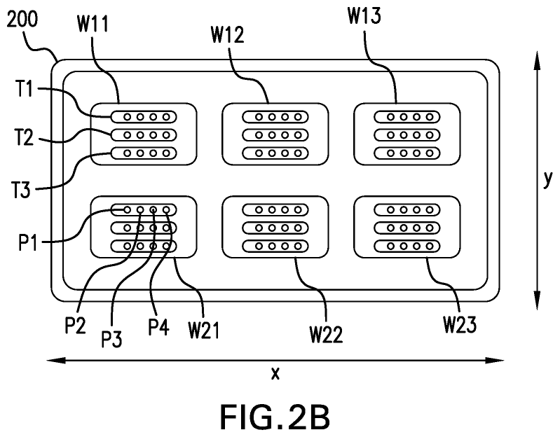

To properly store, align, process and/or identify the organoids, a stationary trough chip 200 may be used. Turning to FIG. 2A, stationary trough chip 200 may include a generally rectangular body 201 having a plurality of wells 202 arranged in rows aligned along the x-axis and/or columns aligned along the y-axis mounted to a transparent plastic or glass support 210 as shown in FIG. 2A. Each well may include a plurality of troughs 204. Body 201 of stationary trough chip 200 may by manufactured by adding a material to a mold 205 (e.g., a stainless-steel mold), via additive manufacturing, or any other suitable manufacturing technique such as injection molding or stamping. The body 201 formed by the mold 205 may be removed from the mold 205 and disposed on a glass support 210 to provide stationary trough chip 200.

Body 201 of stationary trough chip 200 may be manufactured from a suitable material (e.g., a rigid or semi-rigid material) and may be mounted to a glass slide 210. In some alternative embodiments, the body 201 may be formed of glass. In some embodiments, the body is formed from an elastomer. The elastomer in some embodiments is polydimethylsiloxane (PDMS). In some embodiments, the body is formed from polysilsesquioxane, which in further embodiments may be a thermosetting polysilsesquioxane resin. The polysilsesquioxane resin in some embodiments is polymethylsilsesquioxane.

In the example shown, stationary trough chip 200 includes two rows and three columns of wells for a total of six wells formed in the body. The wells may be spaced apart by about 10 mm and 50 mm center-to-center. Each of the wells 202 may include rows of elongated, generally rectangular troughs 204. In the example shown, each well 202 includes three elongated troughs 204. The dimensions of the troughs may be varied, but in one example, the troughs are 600 μm in length and 500 μm deep, and are configured to contain organoids for growth and injection. For example, each trough may be configured and arrange to hold between one or more organoids, in particular embodiments, about seven organoids. The micro-troughs may be spaced from one another from about 1 mm and 5 mm center-to-center. Organoids may be seeded into the troughs in a semi-solid substrate structure (e.g., Corning® Matrigel®), which may be useful for organoid growth and for keeping the organoids in a static position. The seeded organoids are typically incubated in a semi-solid substrate comprising a growth medium suitable for growing or propagating the particular organoid type being cultivated. In this example, the organoids may be held single file in one row within each trough and seated on the same Z-plane. Specifically, the shapes and dimensions of the troughs limit movement of the organoids, and prevent the organoids from being repositioned within the troughs. Because the trough chip is generally planar, microinjections may need only substantial movement along the x-axis and/or y-axis.

In use, stationary trough chip 200 may be set on an LED backlight, which in turn is mounted to a motorized X-Y axis stage 104 and platform 105. In some embodiments, the stationary trough chip and/or stage or backlight may have features (e.g., clips, pins, etc.) to securely affix the stationary trough chip in position and allow it to move with the stage. Video camera 102 may be mounted above stationary trough chip 200 on a Z-axis motor, which provides control for camera focus. A commercial micromanipulator 106 (e.g., Eppendorf® Injectman®) may be mounted such that at least the tip 316 of the capillary needle 302 is within the view of the video camera frame. In the example described, X-Y stage 104 is utilized to move between different wells of the stationary trough chip and/or to specific troughs within a well while camera 102 and micromanipulator 106 stay in a fixed position above the stationary trough chip. However, other variations of these techniques are possible.

Software may be used (e.g., Lab VIEW® or other suitable operating software) to provide the user with controls for the individual modules (e.g. motor actuation for camera focus along the Z-plane, microinjector, X-Y stage movement along the platform) and a semi-automated microinjection routine which move the capillary needle slightly toward an organoid for injection. For this coordinated routine, the user may frame out (e.g., select, highlight, or otherwise choose) the target organoid on the video camera feed window and trigger the injection. A series of movements for the capillary needle are calculated and executed to approach, pierce, inject, and depart the organoid. Information related to the location and size of the organoid, as well as injection parameters, are logged to a database for future reference or recall. This location information may include a stationary trough chip identifier (e.g., serial number, color, experiment number, etc.). Referring to FIG. 2B, the location information may also include the number and/or location of the well in the stationary trough chip body 201 (i.e., a well in the second row and third column, or simply “W23” following a predetermined two-digit naming scheme where the row number and the column number identify the well), the number and location of the trough (e.g., the second trough within the well or simply “T2”), and the position within a trough (e.g., third position in the trough or simply “P3”). For example, C340-W23-T2-P3 may designate an organoid in stationary chip #340, in a well (W23) in the second row and third column of the stationary trough chip body 201, in the second trough of that well (T2) and in the third position (P3) in the stationary trough chip body 201. With the identified stationary trough chip 200 locked into a slide holder 320 disposed on stage 104 and disposed adjacent to micromanipulator 106 and below capillary needle 302 as shown in FIG. 1 and FIG. 3 an injection routine may be carried out.

In further embodiments, the control of the X-Y stage, the video camera, the micromanipulator, and microinjector may be achieved by software commercially available to enable control of each to be performed on a computer having input means and a graphic user interface (GUI) display monitor. The software further enables the tracking of the organoids and stationary trough chips, providing images of the organoids at different time points, and data concerning the organoids.

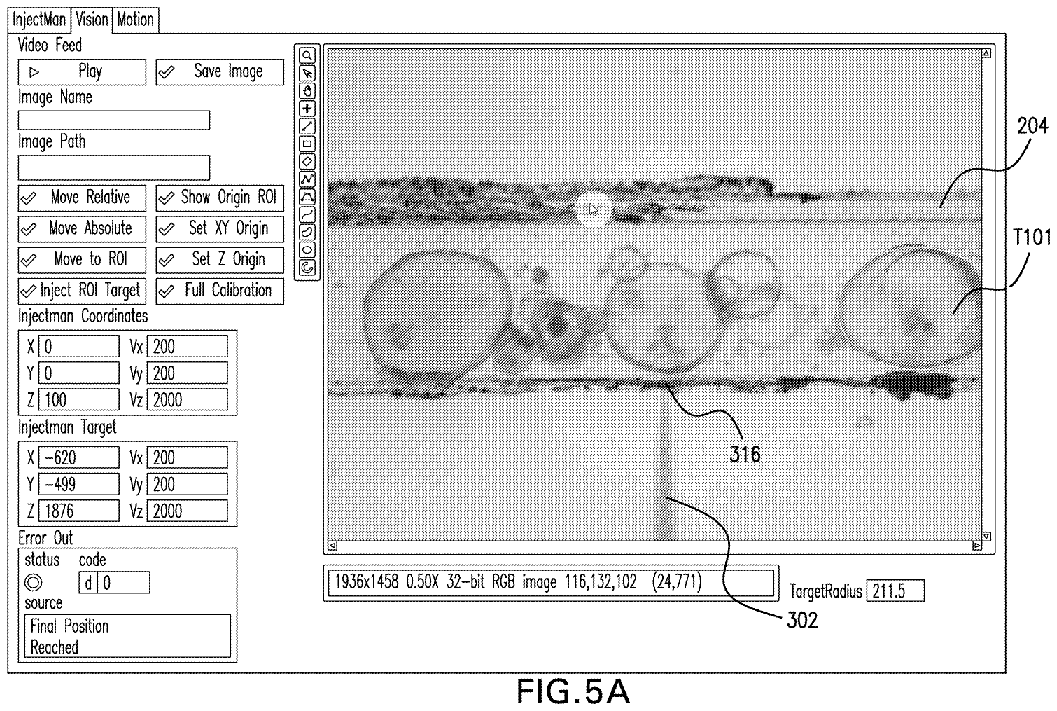

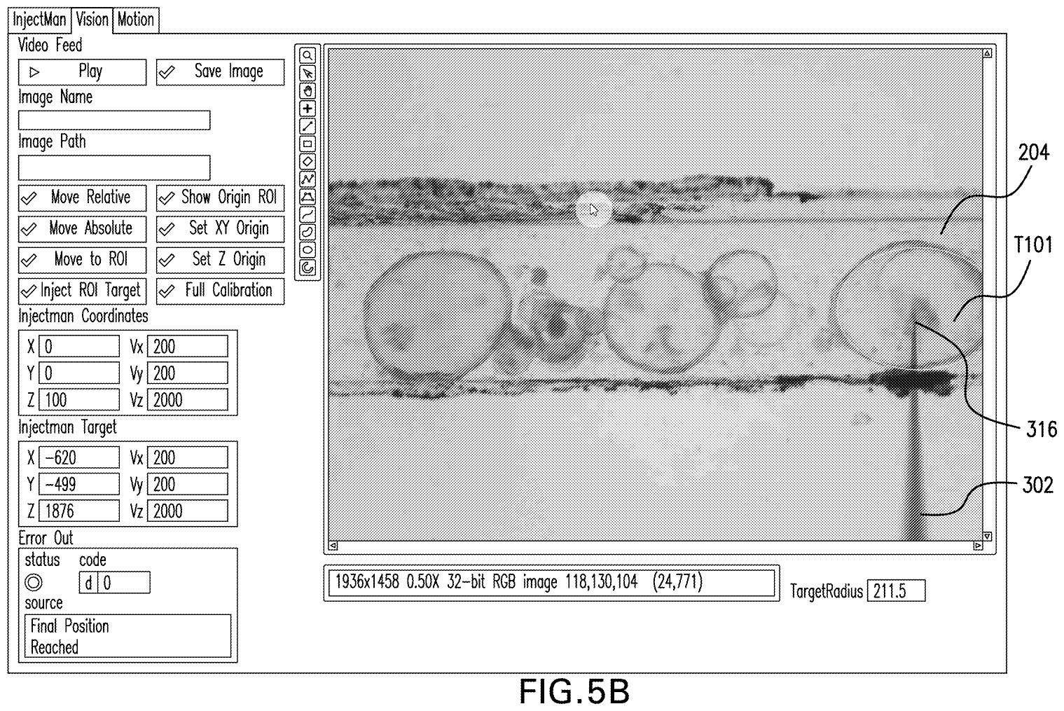

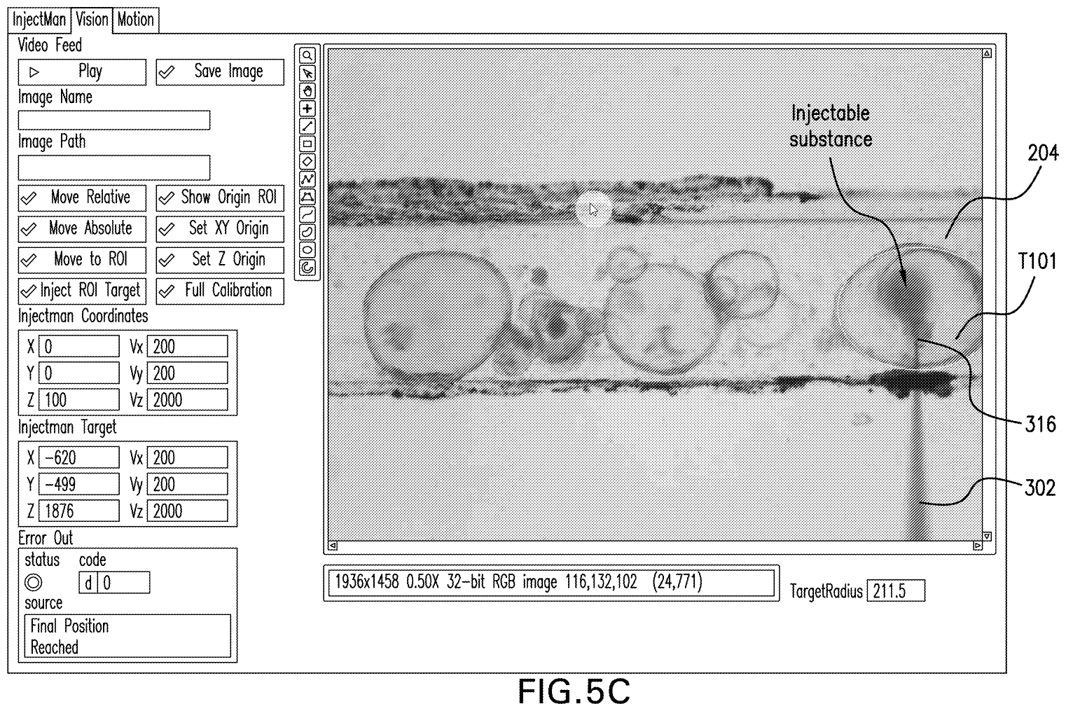

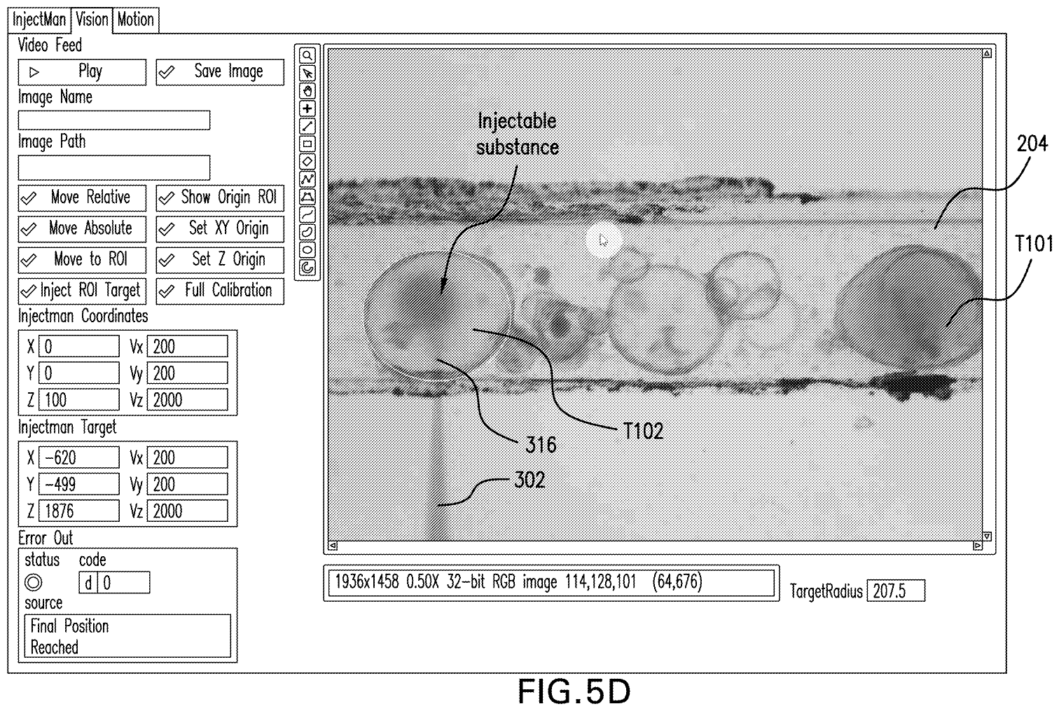

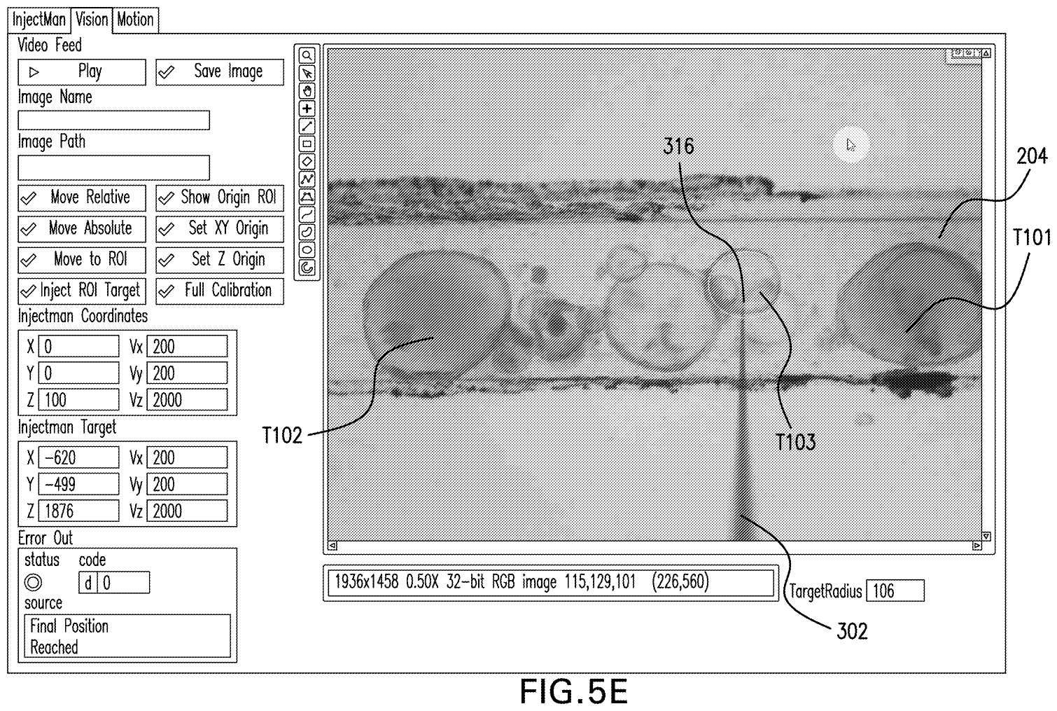

FIGS. 5A-E illustrate a series of screenshots from a GUI display showing captured 4X camera magnified images of a microinjection system routine. As shown within the video camera view frame, a capillary needle 302 and tip 316 is initially in the center position of a trough 204 that has been brought into video camera view frame and the user may use a computer or laptop comprising the operating software to select the target organoid within a trough of a well within the video camera view frame for injection (FIG. 5A). In this case, a first target organoid T101 is selected by creating a circle around it using a mouse, touchscreen, or other input device (as shown in FIG. 5A). Once selected, the capillary needle 302 and tip 316 may be moved using the micromanipulator so that target organoid T101 aligns with the capillary needle 302 and tip 316 and is urged to pierce the organoid (FIG. 5B) and the microinjector activated to inject a predetermined amount of injectable substance into the organoid as shown in FIG. 5C. In these examples, the X-Y stage holding the organoids moves while the capillary needle remains stationary until the desired video camera view is brought into frame and the capillary needle is then moved for alignment with a target organoid within the video camera frame view. However, it will be understood that in an alternative embodiment, the X-Y stage may remain in a fixed position while the capillary needle and video camera are moved in concert.

The injected organoid T101 may be identified from its coloring or shading in those embodiments in which a dye or light refractive material is added to the injectable substance. Information about the substance being injected, FemtoJet® parameters, the location of the organoid (e.g., the well number and/or location, the trough number and/or location, and the position within the trough) may be logged and documented on the computer. The user may then select a second target organoid T102 as before, and the organoid is aligned with the capillary needle as before and the needle is urged to pierce the second target organoid T102 and the microinjector activated to release a predetermined amount of injectable substance into the organoid (FIG. 5D). This process of selecting a target organoid, moving the X-Y stage and/or the capillary needle to the desired position and injecting the organoid may be repeated until all the desired organoids are injected (e.g., T101, T102, T103, etc.) (FIG. 5E). This information may be logged and stored on a computer or portable storage system and retrieved whenever it is needed. Once the microinjection routine is completed, stationary trough chip 200 may be removed from the X-Y stage and sent away for storage or further processing. A second stationary trough chip may be disposed on or coupled to the X-Y stage, and a new microinjection routine may be carried out using the same or different substances or stimuli (e.g. bacteria, antibody, small molecule, damage/repair agent, etc.).

It is to be understood that the embodiments described herein are merely illustrative of the principles and applications of the present disclosure. For example, the number, positioning, spacing and/or arrangement of the wells of the stationary trough chip may be varied. Likewise, the number, positioning, spacing and/or arrangement of the troughs within the wells may be varied. Additionally, the materials of the stationary trough chip may be changed as desired.

Movement of the stationary trough chip and/or the capillary needle may also be varied as discussed in this disclosure to achieve accurate and proper injection. Various techniques may be used to log the information relating to the injections, or to store them or communicate them to a desktop CPU, laptop, cloud-based servers, etc. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A stationary trough chip for microinjection of organoids, comprising: a body including a series of wells, each of the wells having a plurality of troughs disposed therein.

2.-10. (canceled)

11. The stationary trough chip for microinjection of claim 1, wherein each of the plurality of troughs contains a semi-solid substrate structure for seeding the organoids.

12. A method of organoid microinjection, comprising:
(a) providing a microinjection system comprising an X-Y stage, a capillary needle having a tip containing an injectable substance, and a video camera positioned above the X-Y stage;
(b) providing a stationary trough chip comprising a body including a plurality of wells arranged in rows and columns, each of the plurality of wells having a plurality of troughs and a plurality of organoids disposed in at least one of the troughs;
(c) placing the stationary trough chip on the X-Y stage;
(d) selecting a first organoid in a first trough of the stationary trough chip;
(e) moving at least one of the X-Y stage and the capillary needle to align the tip of the capillary needle with the first organoid; and
(f) piercing the first organoid and injecting it with the injectable substance.

13. The method of claim 12, wherein moving at least one of the X-Y stage and the capillary needle to align the tip of the capillary needle with the first organoid includes moving the X-Y stage along a plane in which the stationary trough chip is disposed.

14. The method of claim 12, wherein moving at least one of the X-Y stage and the capillary needle to align the tip of the capillary needle with the first organoid includes moving the capillary needle along a plane in which the stationary trough chip is disposed.

15. The method of claim 12, further comprising a step of selecting a second organoid in the first trough of the stationary trough chip, moving at least one of the X-Y stage and the capillary needle to align the tip of the capillary needle with the second organoid, piercing the second organoid with the tip of capillary needled, and injecting it with the substance.

16. The method of claim 15, wherein the second organoid is injected with the same injectable substance that was injected into the first organoid or wherein the second organoid is injected with a second injectable substance that is different from the injectable substance injected into the first organoid.

17. (canceled)

18. The method of claim 12, further comprising the step of logging information on the first substance and the location of the first organoid.

19. The method of claim 12, wherein providing a stationary trough chip comprising a body includes providing a stationary trough chip having six wells.

20. The method of claim 12, wherein providing a stationary trough chip comprising a body includes providing a body having wells with each well including three troughs aligned in a column.

21. A microinjection system comprising:
(a) a stationary trough chip comprising a series of wells, each of the wells having a plurality of troughs disposed therein;
(b) an apparatus having an X-Y stage having for receiving the stationary trough chip, the stage being translatable along an X axis and a Y axis defining an X-Y plane;
(c) a capillary needle having a tip and a lumen therethrough open at both ends, which is positioned above the stationary trough chip; and,
(d) a video camera positioned above the X-Y stage and capable of recording a video stream comprising a frame view of a trough and the tip of the capillary needle, the video camera translatable along a Z-axis perpendicular to the X-Y stage.

22. The microinjection system of claim 21, wherein each of the plurality of troughs is approximately 500 μm deep.

23. The microinjection system of claim 22, wherein each of the plurality of troughs is approximately 600 μm in length.

24. The microinjection system of claim 21, wherein the capillary needle is provided by a micromanipulator comprising a capillary holder having a distal end and a proximal end and a lumen therethrough open at both ends in which the capillary needle is releasably gripped by the distal end of the capillary holder and wherein the micromanipulator enables the translation of the capillary needle along the X, Y, and Z axes.

25. The microinjection system of claim 24, wherein an injection tubing having a distal end and a proximal end is provided wherein the distal end of the injection tubing is releasably gripped by the proximal end of the capillary holder and the proximal end of the injection tubing is releasably connected to a microinjector capable of providing precise and reproducible injections of femtoliter to microliter amounts of fluids.

26. In a microinjection system comprising an apparatus having an X-Y stage, the stage being translatable along an X axis and a Y axis defining an X-Y plane; a capillary needle having a tip and a lumen therethrough open at both ends; and, a video camera positioned above the X-Y stage and capable of recording a video stream comprising a frame view of X-Y stage, the video camera translatable along a Z-axis perpendicular to the X-Y stage, the improvement comprising
a stationary trough chip comprising a series of wells, each of the wells having a plurality of troughs disposed therein for receiving a multiplicity of organoids, wherein the organoids are positioned within the trough in a row on the same plane and the stationary trough chip is positioned on the X-Y stage.

27. The microinjection system of claim 26, wherein each of the plurality of troughs is approximately 500 μm deep.

28. The microinjection system of claim 27, wherein each of the plurality of troughs is approximately 600 μm in length.

29. The microinjection system of claim 26, wherein the capillary needle is provided by a micromanipulator comprising a capillary holder having a distal end and a proximal end and a lumen therethrough open at both ends in which the capillary needle is releasably gripped by the distal end of the capillary holder and wherein the micromanipulator enables the translation of the capillary needle along the X, Y, and Z axes.

30. The microinjection system of claim 29, wherein an injection tubing having a distal end and a proximal end is provided wherein the distal end of the injection tubing is releasably gripped by the proximal end of the capillary holder and the proximal end of the injection tubing is releasably connected to a microinjector capable of providing precise and reproducible injections of femtoliter to microliter amounts of fluids.
